(19) United States
(12) Patent Application Publication
(10) Pub. No.: US 2024/0127928 A1
(43) Pub. Date: Apr. 18, 2024

(54) EFFECTUATING ABNORMAL BONE CURVATURE TREATMENT USING GAN

(71) Applicant: INTERNATIONAL BUSINESS MACHINES CORPORATION, ARMONK, NY (US)

(72) Inventors: Sarbajit K. Rakshit, Kolkata (IN); Sathya Santhar, Ramapuram (IN); Sridevi Kannan, Chennai (IN)

(21) Appl. No.: 18/047,293

(22) Filed: Oct. 18, 2022

(51) Int. Cl. G16H 30/20; G06N 3/094

(52) U.S. Cl. CPC G16H 30/20; G06N 3/094

(57) ABSTRACT

A method of effectuating abnormal bone curvature treatment using a generative adversarial network (GAN) is provided. The method includes training the GAN using treatment data for each of a plurality of abnormal bone curvature patients. The treatment data for each abnormal bone curvature patient includes a plurality of radiologic images taken during at least a partial treatment period and at least one orthotic used during the at least partial treatment period. An abnormal bone curvature is identified in a radiologic image for a new patient using the trained GAN. A plurality of artificial radiologic images is generated corresponding to different points in a treatment period of the new patient using the identified abnormal bone curvature and the corresponding radiologic image for the new patient as inputs to the trained GAN.

### BACKGROUND

Exemplary embodiments of the present inventive concept relate to abnormal bone curvature treatment, and more particularly, to effectuating abnormal bone curvature treatment using a generative adversarial network (GAN).

GANs are algorithmic architectures that use two neural networks, pitting one against the other (thus the “adversarial” aspect) to generate new, synthetic instances of data that can pass for real data. They are used widely in synthetic image, video, and voice generation.

### SUMMARY

Exemplary embodiments of the present inventive concept relate to a method, a computer program product, and a system for effectuating abnormal bone curvature treatment using a GAN.

According to an exemplary embodiment of the present inventive concept, a method of effectuating abnormal bone curvature treatment using a generative adversarial network (GAN) is provided. The method includes training the GAN using treatment data for each of a plurality of abnormal bone curvature patients. The treatment data for each abnormal bone curvature patient includes a plurality of radiologic images taken during at least a partial treatment period and at least one orthotic used during the at least partial treatment period. An abnormal bone curvature is identified in a radiologic image for a new patient using the trained GAN. A plurality of artificial radiologic images is generated corresponding to different points in a treatment period of the new patient using the identified abnormal bone curvature and the corresponding radiologic image for the new patient as inputs to the trained GAN.

According to an exemplary embodiment of the present inventive concept, a computer program product for effectuating abnormal bone curvature treatment using a generative adversarial network (GAN) is provided. The computer program product includes one or more computer-readable storage media and program instructions stored on the one or more non-transitory computer-readable storage media capable of performing a method. The method includes training the GAN using treatment data for each of a plurality of abnormal bone curvature patients. The treatment data for each abnormal bone curvature patient includes a plurality of radiologic images taken during at least a partial treatment period and at least one orthotic used during the at least partial treatment period. An abnormal bone curvature is identified in a radiologic image for a new patient using the trained GAN. A plurality of artificial radiologic images is generated corresponding to different points in a treatment period of the new patient using the identified abnormal bone curvature and the corresponding radiologic image for the new patient as inputs to the trained GAN.

According to an exemplary embodiment of the present inventive concept, a computer system for effectuating abnormal bone curvature treatment using a generative adversarial network (GAN) is provided. The computer system includes one or more computer processors, one or more computer-readable storage media, and program instructions stored on the one or more of the computer-readable storage media for execution by at least one of the one or more processors capable of performing a method. The method includes training the GAN using treatment data for each of a plurality of abnormal bone curvature patients. The treatment data for each abnormal bone curvature patient includes a plurality of radiologic images taken during at least a partial treatment period and at least one orthotic used during the at least partial treatment period. An abnormal bone curvature is identified in a radiologic image for a new patient using the trained GAN. A plurality of artificial radiologic images is generated corresponding to different points in a treatment period of the new patient using the identified abnormal bone curvature and the corresponding radiologic image for the new patient as inputs to the trained GAN.

### BRIEF DESCRIPTION OF THE DRAWINGS

The following detailed description, given by way of example and not intended to limit the exemplary embodiments solely thereto, will best be appreciated in conjunction with the accompanying drawings, in which:

FIG. 1 illustrates a schematic diagram of computing environment 100, which may be used for abnormal bone curvature treatment using a GAN, in accordance with an exemplary embodiment of the present inventive concept.

FIG. 2 illustrates a flowchart of abnormal bone curvature treatment using a GAN 200, in accordance with an exemplary embodiment of the present inventive concept.

FIG. 3 illustrates a block diagram of the system of abnormal bone curvature treatment using a GAN 200, according to an exemplary embodiment of the present invention.

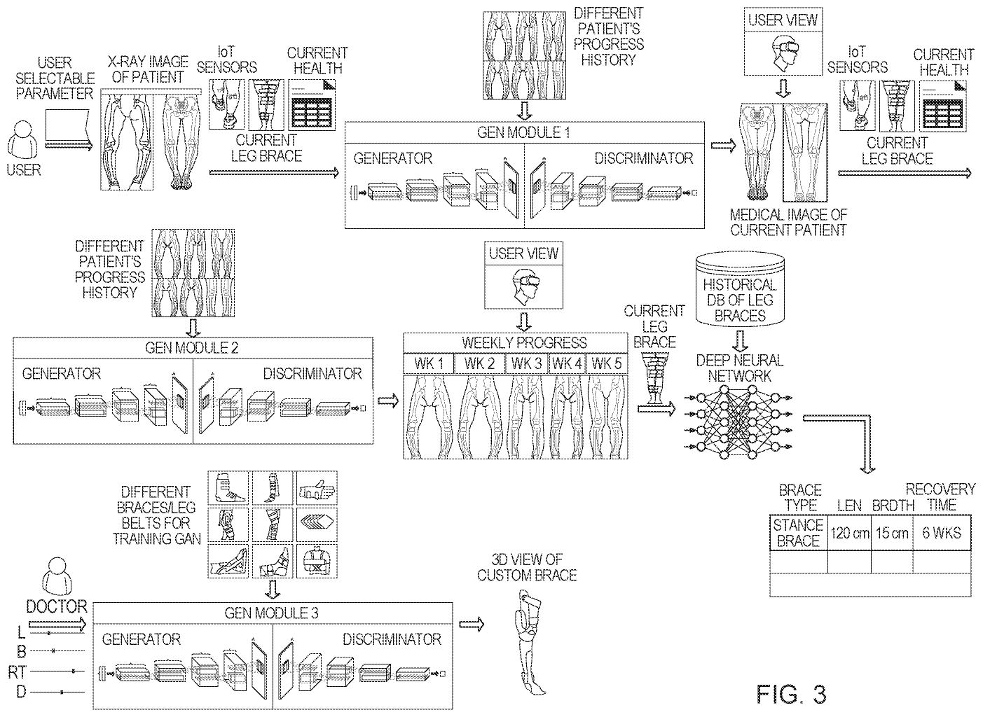

It is to be understood that the included drawings are not necessarily drawn to scale/proportion. The included drawings are merely schematic examples to assist in understanding of the present inventive concept and are not intended to portray fixed parameters. In the drawings, like numbering may represent like elements.

### DETAILED DESCRIPTION

Exemplary embodiments of the present inventive concept are disclosed hereafter. However, it shall be understood that the scope of the present inventive concept is dictated by the claims. The disclosed exemplary embodiments are merely illustrative of the claimed system, method, and computer program product. The present inventive concept may be embodied in many different forms and should not be construed as limited to only the exemplary embodiments set forth herein. Rather, these included exemplary embodiments are provided for completeness of disclosure and to facilitate an understanding to those skilled in the art. In the detailed description, discussion of well-known features and techniques may be omitted to avoid unnecessarily obscuring the presented exemplary embodiments.

References in the specification to “one embodiment,” “an embodiment,” “an exemplary embodiment,” etc., indicate that the embodiment described may include a particular feature, structure, or characteristic, but not every embodiment may necessarily include that feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether explicitly described.

In the interest of not obscuring the presentation of the exemplary embodiments of the present inventive concept, in the following detailed description, some processing steps or operations that are known in the art may have been combined for presentation and for illustration purposes, and in some instances, may have not been described in detail. Additionally, some processing steps or operations that are known in the art may not be described at all. The following detailed description is focused on the distinctive features or elements of the present inventive concept according to various exemplary embodiments.

Abnormal bone curvature (e.g., scoliosis, bowleg, etc.) can cause bodily deformation and often has an onset during adolescence. Abnormal bone curvature requires prompt and appropriate treatment to thwart permanence or mitigate severity. Such prompt and appropriate treatment will reduce associated pain, improper anatomical force absorption, and inefficient movement potentially for a patient's entire life. However, abnormal curvature in one region of a bone may create the appearance of an abnormal curvature in another part of the bone and/or region. A doctor must accurately identify which part of the bone is abnormally curved (causing the apparent deformity), the degree of curvature, three-dimensional orientation thereof, and prescribe the proper treatment protocol. Treatment of abnormal bone curvature may include the use of surgery and/or orthotics such as braces, belts, etc. Orthotics require proper selection, sizing, gradual adjustment, monitoring, and considered timing. Thus, a method to accurately identify abnormal bone curvature, select the appropriate orthotics (or combinations thereof), and develop a prediction/visualization of treatment progress using the individual patient's particular anatomy is needed. The present inventive concept accurately identifies each instance of abnormal bone curvature, generates artificial radiologic images corresponding to a predicted treatment timeline, and determines at least one orthotic to effectuate treatment based on customizable parameters. In addition to enabling more accurate, tailored, expeditious, and reliable patient outcomes, the present inventive concept will also reduce insurance costs, patient out-of-pocket costs, and abnormal bone curvature treatment duration.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as abnormal bone curvature treatment using GAN program 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

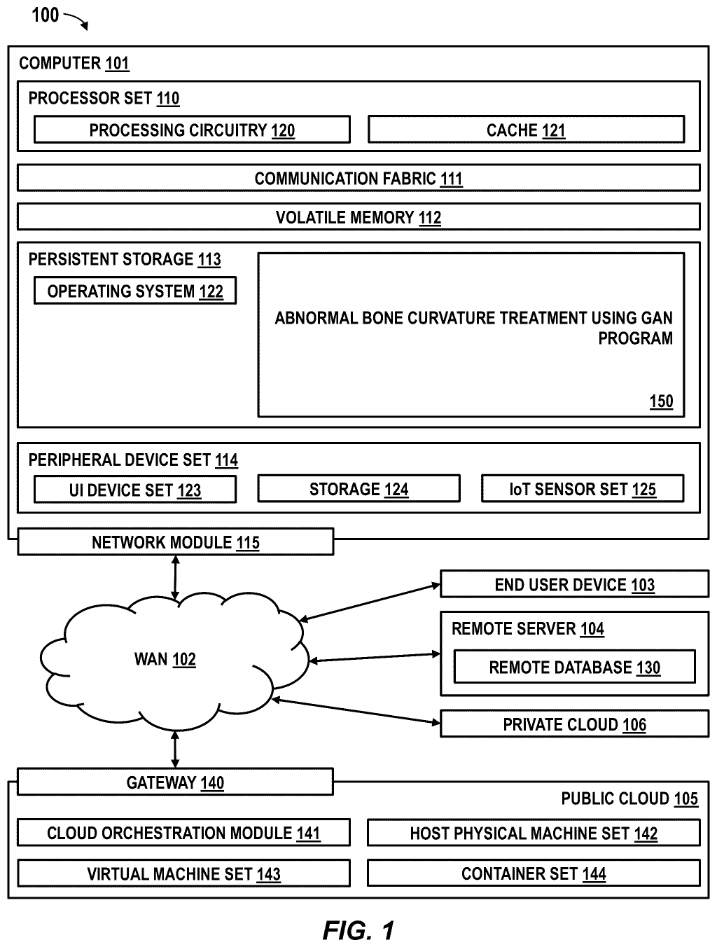

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

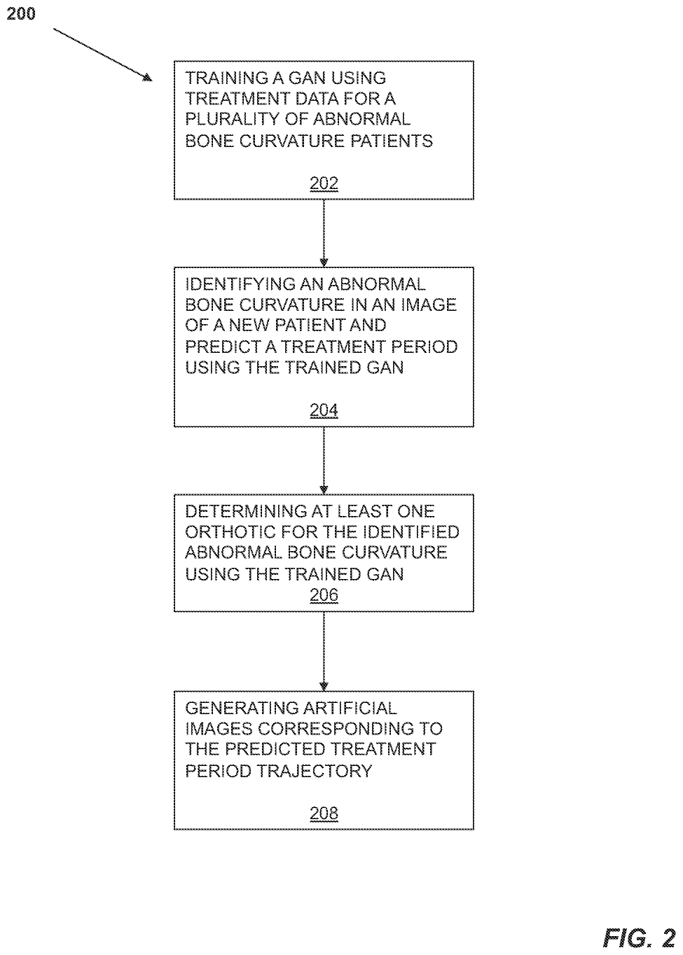

FIG. 2 illustrates a flowchart of abnormal bone curvature treatment using a GAN 200, in accordance with an exemplary embodiment of the present inventive concept.

The abnormal bone curvature treatment using a GAN program 150 may obtain treatment data for a plurality of abnormal bone curvature patients to train the GAN (step 202). The treatment data may include treatment multimedia, such as radiologic imaging (e.g., MRIs, x-rays, etc.) and reports thereof, IoT body sensors, plain imaging illustrative of abnormal bone curvature and reports thereof, non-image pathological reports, diagrams, medical illustrations, dictated notes, patient specific characteristics (e.g., demographics, height, weight, age, bone lengths, activities, medical history, medications, comorbidities, onset of abnormal bone curvature, climate/weather in area of residence, etc.), orthotics (e.g., type, brand, materials, co-use, anatomical placement, dimensions, timing of usage, etc.), billing invoices, and/or respective treatment periods. In an embodiment, the abnormal bone curvature treatment using a GAN program 150 may additionally obtain treatment data via the network regarding an abnormal bone curvature generally (e.g., prognoses, etiology, exacerbating factors, etc.) by performing a keyword search (e.g., diagnosed condition). The treatment multimedia may be obtained by user input (e.g., doctor, patient, etc.) and/or obtained via the network automatically by accessing an authorized patient history repository (e.g., patient portal, database, collective medical care repository, etc.).

The treatment multimedia may be analyzed by machine learning processes (e.g., computer vision, natural language processing (NLP), speech-to-text, etc.). The abnormal bone curvature treatment using a GAN program 150 may determine patterns in abnormal bone curvature patients' analyzed treatment data (e.g., precipitating/exacerbating factors, onset age, treatment onset, orthotics used for particular abnormal bone curvature types and dimensions thereof, duration of treatment periods, effectiveness, confounding diagnoses, comorbidities, etc.). Based on the determined patterns in the analyzed patients' treatment data, the GAN may learn to identify abnormal bone curvature and attributes of diagnosis (e.g., type, degree of curvature departure, plane of curvature, etc.), gain the capacity to produce artificial radiologic images, predict treatment timelines, determine and/or artificially generate orthotics and dimensions thereof, treatment advisories, etc.

For example, the abnormal bone curvature treatment using a GAN program 150 receives treatment data for a plurality of abnormal bone curvature patients via upload from clinicians and clinics specializing in treatment for bowleg adolescents. Computer vision is used to analyze radiologic imaging and progression for each patient during their respective treatment periods as well as abnormal bone curvature and bone length characteristics. NLP and speech-to-text is used to analyze digital medical records for related patient imaging reports, clinical notes, orthotics used, height, weight, age, time of treatment initiation, activities, contributory factors, etc.

The abnormal bone curvature treatment using a GAN program 150 may identify an abnormal bone curvature in at least one image for a new patient and predict a treatment period using the trained GAN using the trained GAN (step 204). The at least one image and/or the patient treatment data for the new patient may be obtained via the network or user input. The at least one image may include at least one abnormal bone curvature and may include a plain image (e.g., taken by a point-and-shoot or digital camera) and/or a radiologic image. The abnormal bone curvature treatment using a GAN program 150 may identify bone dimensions and/or the at least bone curvature using the trained GAN based on similarities with the plurality of prior patients' treatment data (e.g., radiologic images, plain images, patient specific characteristics, etc.). The abnormal bone curvature treatment using a GAN program 150 may communicate a written, visual, or spoken diagnosis corresponding to the identified abnormal bone curvature(s), bone dimensions, and/or an overall clinical assessment to the user. In an embodiment, the abnormal bone curvature treatment using a GAN program 150 may overlay annotations (e.g., diagnosis, area of curvature, degree of curvature, etiology, plane of curvature, likely onset, prognosis, at least one treatment period and/or range thereof, etc.) onto the provided at least one image for the new patient. The abnormal bone curvature treatment using a GAN program 150 may determine at least one treatment period based on the identified abnormal bone curvature. The at least one treatment period may include a typical learned range, best-case/worst-case scenario timeline, a mean duration, a duration corresponding to an orthotic in present use, and/or a personalized duration based on the new patient's specific treatment data.

For example, the abnormal bone curvature treatment using a GAN program 150 receives a radiologic image for a new baby patient with bowlegs, which is uploaded by a treating clinician. From prior training, the abnormal bone curvature treatment using a GAN program 150 identifies the bowleg diagnosis, bone lengths, an associated severe degree of curvature, and congenital scoliosis. The abnormal bone curvature treatment using a GAN program 150 provides annotations for the treating clinician's review with an explanatory note regarding the abnormal bone curvature and the latent diagnosed scoliosis. Based on prior learning, the average treatment period for a baby exhibiting congenital scoliosis of mild severity is one year, while bowlegs of the severity identified is three to four years.

The abnormal bone curvature treatment using a GAN program 150 may determine at least one orthotic for the identified abnormal bone curvature (step 206). The trained GAN may automatically select the at least one orthotic from the learned repository of orthotics (e.g., based on a most expedient, reliable, typical, and/or effective orthotic) or the orthotic may be selected based on user input parameters (e.g., treatment period duration, breadth, length, daily usage, etc.), approximate or exact. In an embodiment, a sliding scale for each user input criteria may be adjusted by the user from an interface of the abnormal bone curvature treatment using a GAN program 150. If the determined at least one orthotic is substantially similar to the orthotic in present use by the new patient, the GAN may not proceed further and may indicate this occurrence to the user. In addition, the suggested treatment period will remain unaltered. If, however, the determined at least one orthotic is different from the orthotic in present use, or no orthotic is in present use, the abnormal bone curvature treatment using a GAN program 150 may generate a model (e.g., blueprint, schematic, visualization, etc.) of the determined at least one orthotic and update the treatment period accordingly. The determined at least one orthotic may be tailored to the specific patient (e.g., based on activities, user input criteria, bone dimensions, etc.) using a deep neural network (DNN). The abnormal bone curvature treatment using a GAN program 150 may print the model using a connected three-dimensional (3D) printer. In an embodiment, when the abnormal bone curvature treatment using a GAN program 150 has determined a plurality of orthotics are appropriate, the models for the plurality of orthotics may be integrated, proportioned, manufactured, and/or coupled accordingly.

The abnormal bone curvature treatment using a GAN program 150 determines that precise iterations of learned orthotics, such as a brace, are appropriate options to treat the mild congenital scoliosis, but that the severe bowlegs will require a customized orthotic. The patient's family indicates that they would like to initiate aggressive treatment with a rigid orthotic that is worn near constantly. The abnormal bone curvature treatment using a GAN program 150 generates a customized orthotic model to treat the bowlegs in the lower end of the time period range (three years). Once the customized orthotic model is approved, the doctor proceeds to 3D print the customized orthotic device and orders the brace for congenital scoliosis, which is determined to be a lower cost to the patient's family.

The abnormal bone curvature treatment using a GAN program 150 may generate artificial images corresponding to a trajectory of the predicted treatment period (step 208). The abnormal bone curvature treatment using a GAN program 150 may use the GAN which was trained with the plurality of prior patients' radiologic and/or plain images to generate the artificial images (e.g., plain images and/or radiologic images) for the new patient. The abnormal bone curvature treatment using a GAN program 150 may receive the new patient plain and/or radiologic images, new patient treatment data, user input parameters, determined at least one orthotic, and/or the predicted treatment period as inputs. The user may select the depiction frequency (e.g., first/last, daily, weekly, monthly, randomly, etc.) of artificial images generated within the predicted treatment period. The abnormal bone curvature treatment using a GAN program 150 may overlay annotations (e.g., based on the depiction frequency) which provide clinically significant insights (e.g., percentage of abnormal curvature correction effectuated, cautions at certain junctures, etc.) and/or demarcate the area of curvature and/or indirectly effected bone regions. In addition, the abnormal bone curvature treatment using a GAN program 150 may generate and/or modify a series of artificial plain images corresponding to the predicted treatment period with at least one orthotic (e.g., the orthotic in present use or an otherwise determined orthotic) and/or incremental adjustments/changes therefor. In an embodiment, the abnormal bone curvature treatment using a GAN program 150 may periodically run a comparison analysis between the new patient's present imaging and the generated artificial images to detect discrepancies and suggest ameliorative action (e.g., modifying/changing orthotics, evaluating causes of trajectory deviation, etc.).

For example, the patient's family indicates that they would like the frequency of artificial images generated to coincide with their monthly appointments. The abnormal bone curvature treatment using a GAN program 150 thus generates a monthly series of artificial radiologic and plain images spanning the projected three-year bowleg treatment period (with annotations at each juncture) based on training gained from imitating the plurality of prior patients, in particular, those of similar condition and/or age. Because the scoliosis identified was mild, the family of the patient decide to forgo the expense of generating artificial radiologic images. One year later, the family again presents to the clinician for a radiologic image and follow-up. The mild congenital scoliosis is cured, as anticipated, however, the bowlegs persist and are a month beyond in the treatment period trajectory based on comparison to the artificial radiologic imaging. The abnormal bone curvature treatment using a GAN program 150 associates an increase in the patient's weight, height, activity, and probable leniency with strict use as explaining this finding, and suggests modifications accordingly.

In an embodiment, the GAN referenced herein may be a modified conditional GAN. A generator of the GAN may be modified to receive conditional inputs (such as a radiologic image of the patient including an abnormal bone curvature) and might not generate a new, novel artificial radiologic image altogether, but instead may use the conditional input as a base image and will provide a new artificial radiologic image (without any abnormal bone curvature) based on the base image and historical data. During a training phase, the generator may be trained to receive the base image as a conditional input, and a discriminator of the GAN may be trained to reject the image if the new artificial radiologic image has no correlation with the input base image. Hence the generator (G) over historical data (D) may build a mapping function from a noise distribution space (Pn) and also on conditional input space (C). The loss function for the GAN may be reduced, which is a combination of loss with respect to the conditioned base image and the loss with respect to historical data.

Based on the foregoing, a computer system, method, and computer program product have been disclosed. However, numerous modifications, additions, and substitutions can be made without deviating from the scope of the exemplary embodiments of the present inventive concept. Therefore, the exemplary embodiments of the present inventive concept have been disclosed by way of example and not by limitation.

1. A method of effectuating abnormal bone curvature treatment using a generative adversarial network (GAN), the method comprising:
training the GAN using treatment data for each of a plurality of abnormal bone curvature patients, wherein the treatment data for each abnormal bone curvature patient includes a plurality of radiologic images taken during at least a partial treatment period and at least one orthotic used during the at least partial treatment period;
identifying an abnormal bone curvature in a radiologic image for a new patient using the trained GAN; and
generating a plurality of artificial radiologic images corresponding to different points in a treatment period of the new patient using the identified abnormal bone curvature and the corresponding radiologic image for the new patient as inputs to the trained GAN.

2. The method of claim 1, further comprising:
determining at least one orthotic for the identified abnormal bone curvature using the trained GAN and an associated treatment period for the at least one determined orthotic.

3. The method of claim 2, further comprising:
generating a plurality of artificial radiologic images corresponding to different points in the associated treatment period for the at least one determined orthotic using the identified abnormal bone curvature and the corresponding radiologic image for the new patient as inputs to the trained GAN.

4. The method of claim 1, wherein the treatment period of the new patient is associated with a prescribed orthotic.

5. The method of claim 3, wherein the determined orthotic is a visualization of an artificial orthotic generated by the trained GAN.

6. The method of claim 5, wherein the artificial orthotic visualization and the associated treatment period are based on user input variables, and wherein the user input variables include length, breadth, recovery time, and usage/day.

7. The method of claim 2, wherein the determined orthotic is based on the fastest recovery outcome or the most consistent recovery outcome.

8. A computer program product for effectuating abnormal bone curvature treatment using a generative adversarial network (GAN), the computer program product comprising:
one or more computer-readable storage media and program instructions stored on the one or more non-transitory computer-readable storage media capable of performing a method, the method comprising:
training the GAN using treatment data for each of a plurality of abnormal bone curvature patients, wherein the treatment data for each abnormal bone curvature patient includes a plurality of radiologic images taken during at least a partial treatment period and at least one orthotic used during the at least partial treatment period;
identifying an abnormal bone curvature in a radiologic image for a new patient using the trained GAN; and
generating a plurality of artificial radiologic images corresponding to different points in a treatment period of the new patient using the identified abnormal bone curvature and the corresponding radiologic image for the new patient as inputs to the trained GAN.

9. The computer program product of 8, further comprising:
determining at least one orthotic for the identified abnormal bone curvature using the trained GAN and an associated treatment period for the at least one determined orthotic.

10. The computer program product of claim 9, further comprising:
generating a plurality of artificial radiologic images corresponding to different points in the associated treatment period for the at least one determined orthotic using the identified abnormal bone curvature and the corresponding radiologic image for the new patient as inputs to the trained GAN.

11. The computer program product of claim 8, wherein the treatment period of the new patient is associated with a prescribed orthotic.

12. The computer program product of claim 10, wherein the determined orthotic is a visualization of an artificial orthotic generated by the trained GAN.

13. The computer program product of claim 12, wherein the artificial orthotic visualization and the associated treatment period are based on user input variables, and wherein the user input variables include length, breadth, recovery time, and usage/day.

14. The computer program product of claim 9, wherein the determined orthotic is based on the fastest recovery outcome or the most consistent recovery outcome.

15. A computer system for effectuating abnormal bone curvature treatment using a generative adversarial network (GAN), the computer system comprising:
one or more computer processors, one or more computer-readable storage media, and program instructions stored on the one or more of the computer-readable storage media for execution by at least one of the one or more processors capable of performing a method, the method comprising:
training the GAN using treatment data for each of a plurality of abnormal bone curvature patients, wherein the treatment data for each abnormal bone curvature patient includes a plurality of radiologic images taken during at least a partial treatment period and at least one orthotic used during the at least partial treatment period;
identifying an abnormal bone curvature in a radiologic image for a new patient using the trained GAN; and
generating a plurality of artificial radiologic images corresponding to different points in a treatment period of the new patient using the identified abnormal bone curvature and the corresponding radiologic image for the new patient as inputs to the trained GAN.

16. The computer system of claim 15, further comprising:
determining at least one orthotic for the identified abnormal bone curvature using the trained GAN and an associated treatment period for the at least one determined orthotic.

17. The computer system of claim 16, further comprising:
generating a plurality of artificial radiologic images corresponding to different points in the associated treatment period for the at least one determined orthotic using the identified abnormal bone curvature and the corresponding radiologic image for the new patient as inputs to the trained GAN.

18. The computer system of claim 15, wherein the treatment period of the new patient is associated with a prescribed orthotic.

19. The computer system of claim 17, wherein the determined orthotic is a visualization of an artificial orthotic generated by the trained GAN.

20. The computer system of claim 19, wherein the artificial orthotic visualization and the associated treatment period are based on user input variables, and wherein the user input variables include length, breadth, recovery time, and usage/day.
